(19) United States
(12) Patent Application Publication
(10) Pub. No.: US 2005/0069563 A1
(43) Pub. Date: Mar. 31, 2005

(54) Method for treating herpes simplex virus and fungal pimples with antifungal medication

(76) Inventor: Michael D. Garner, Henderson, NV (US)

Correspondence Address: WEISS & MOY PC, 4204 NORTH BROWN AVENUE, SCOTTSDALE, AZ, 85251, US

(21) Appl. No.: 10/672,037

(22) Filed: Sep. 26, 2003

(51) Int. Cl. A61K 31/137; A61K 9/00

(52) U.S. Cl. 424/400; 514/649

(57) ABSTRACT

A method for treating herpes simplex virus wherein an individual applies antifungal medication onto an area of skin that is infected with the virus. And a method for treating fungal pimples by first softening the fungal pimple and then applying antifungal medication to it.

### BACKGROUND OF THE INVENTION

1. Field of the Invention

This invention relates generally to dermatology, and more specifically to a method for treating herpes simplex virus and fungal pimples with antifungal medication.

2. Description of Prior Art

Herpes simplex is a common viral infection that causes oral herpes, and genital herpes. There are two strains of herpes simplex virus, Herpes Simplex Type 1 (HSV-1) and Herpes Simplex Type 2 (HSV-2). HSV-1 usually causes cold sores and fever blisters, whereas HSV-2 typically causes genital sores.

Herpes simplex virus infections are contagious. An infected individual is considered contagious when active sores are present and potentially contagious when no symptoms are present. Transmission of the herpes simplex virus may occur via saliva or by any direct skin-to-skin contact with the infected area.

Herpes simplex virus infections are often recurrent and unfortunately there is no known cure for the herpes simplex virus. At the onset of the infection, the virus enters the nerve endings and travels to the dorsal root ganglia where it remains inactive. Recurrence takes place when the latent virus is triggered by immunosuppressive events, such as stress, sun exposure, menses, or injury to the area. Once it is reactivated, the virus travels back down to the surface of the skin, causing another outbreak.

Cold sores are small, clear, fluid-filled blisters that appear on or near the lips. They will most often disappear on their own within seven to ten days. Although there is no cure for the cold sore-causing HSV-1 virus, several prescription and non-prescription products are available to help relieve the pain and discomfort associated with the cold sores. However, these products do not cause the cold sores to heal any faster. Furthermore, many of these products appear in cream forms which have difficulty in penetrating an individual's intact skin.

The HSV-2 virus often causes genital sores to appear on the buttocks, penis, vagina, or cervix. These genital sores are similar in nature to the cold sores discussed above. Therefore, it is believed that HSV-2 may be treated the same way as HSV-1.

Pimples are small raised bumps that are filled with pus and they may appear anywhere on the body. Pimples are often white-tipped, owing to the white blood cells that are working to fight the inflammation. However, sometimes pimples may be hard and filled with a clear, yellow liquid. Unlike the white blood cells that may be ejected by squeezing the average pimple, these fungal pimples contain a granuloma that remains trapped within the skin. This granuloma causes recurrent outbreaks in the same area. There are many prescription and non-prescription products, such as acne creams, that are available to treat the common pimple. However these creams are not effective on a fungal pimple because it is difficult for the cream to penetrate the hardened layer of skin and these creams may be ineffective against the underlying fungus.

Therefore, a need existed to provide a method for treating herpes simplex virus that will allow medication to penetrate the skin and that will accelerate the healing process of a cold sore.

A further need also existed to provide a method for treating fungal pimples.

### SUMMARY OF THE INVENTION

It is an object of the present invention to provide a method for treating herpes simplex virus that will allow medication to penetrate the skin and that will accelerate the healing process of a cold sore.

It is another object of the present invention to provide a method for treating fungal pimples.

### BRIEF DESCRIPTION OF THE PREFERRED EMBODIMENTS

In accordance with one embodiment of the present invention, a method for treating herpes simplex virus is disclosed. In order to treat herpes simplex virus, an individual should apply antifungal medication onto an area of skin that is infected with the virus.

In accordance with another embodiment of the present invention, a method for treating fungal pimples is disclosed. In order to treat fungal pimples, the individual should first soften the fungal pimple and then apply antifungal medication to the fungal pimple.

The foregoing and other objects, features, and advantages of the invention will be apparent from the following, more particular, description of the preferred embodiments of the invention, as illustrated in the accompanying drawings.

### BRIEF DESCRIPTION OF THE DRAWINGS

The novel features believed characteristic of the invention are set forth in the appended claims. The invention will best be understood by reference to the following detailed description of illustrated embodiments when read in conjunction with the accompanying drawings, wherein like reference numerals and symbols represent like elements.

FIG. 1 is a front view of a cream form and a liquid form of antifungal medication combined to form an amalgam of antifungal medication inside a bottle.

FIG. 2 is a perspective view of an applicator being loaded with the amalgam of antifungal medication.

FIG. 3 is a front view of the amalgam of antifungal medication being applied to a cold sore with the applicator.

FIG. 4 is a perspective view of a fungal pimple on an individual's hand.

FIG. 5 is a perspective view of a softening agent being applied to the fungal pimple with a cotton swab.

FIG. 6 is a perspective view of the softening agent being wiped off of the fungal pimple by a clean cloth.

FIG. 7 is a perspective view of the liquid form of antifungal medication being applied to the fungal pimple.

### DETAILED DESCRIPTION

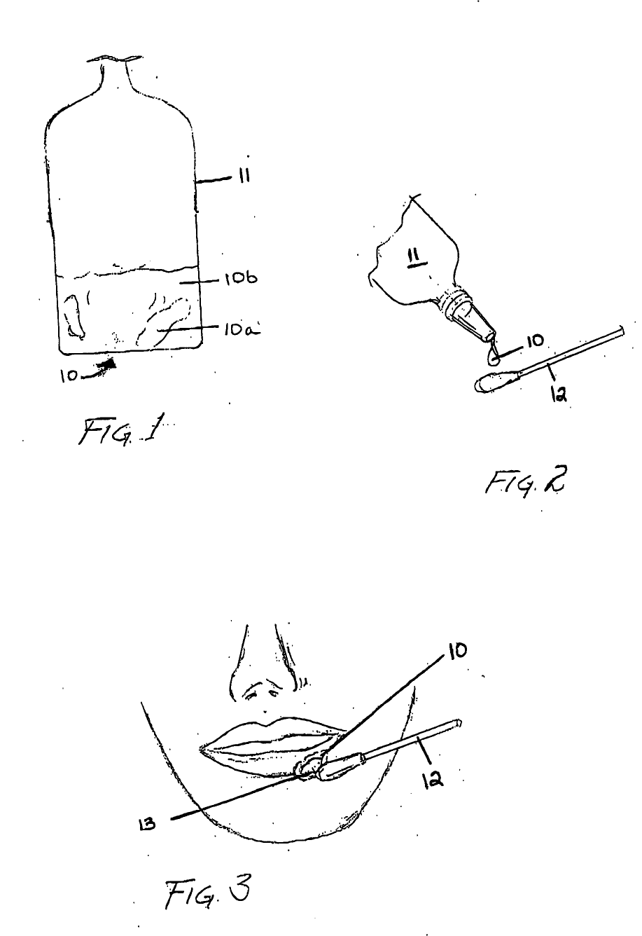

During an individual's immunosuppressed state, fungal reproduction in the body is at an increased level. The inventor believes that it is this high level of fungal reproduction that promotes the recurrence of herpes simplex virus outbreaks. Therefore the inventor has devised a method for treating herpes simplex virus with antifungal medication (see FIGS. 1-5). The antifungal medication used in the preferred embodiments is terbinafine hydrochloride, and more specifically LAMISIL^{AT}, although it should be clearly understood that substantial benefit could be derived from an alternative configuration of the method for treating herpes simplex virus in which another antifungal medication is used. The antifungal medication is available in a substantially cream form 10a (see FIG. 1) and in a substantially liquid form 10b (see FIG. 1).

In treating a cold sore, the inventor has noted that while the substantially cream form 10a is able to penetrate a lip's pseudo mucus membrane, it is unable to penetrate any surrounding intact skin. The substantially liquid form 10b, on the other hand, is able to penetrate intact skin. However, the very nature of the liquid form makes it difficult to control during application. Control during application of antifungal medication is important because antifungal medication is not meant for internal use or ingestion.

FIGS. 1-3 refer to the treatment of a cold sore 13. FIG. 1 depicts an amalgam 10 of antifungal medication created by combining an approximately 1:1 ratio of the substantially cream form 10a and the substantially liquid form 10b of antifungal medication in a bottle 11. As shown in FIG. 2, a predetermined amount of the amalgam 10 of antifungal medication is loaded onto one of an applicator 12 and a cotton swab. And in FIG. 3, the applicator 12 is then used to apply the amalgam 10 of antifungal medication onto the cold sore 13 and surrounding area. This amalgam 10 of antifungal medication may also be applied to a genital sore (not shown). Although, in the preferred embodiment, the antifungal medication applied is in the form of an amalgam 10 of the substantially cream form 10a and the substantially liquid form 10b, it should be clearly understood that substantial benefit could be derived from an alternative embodiment of the present invention in which a cold sore 13 or a genital sore is treated with only the substantially cream form 10a, or only the substantially liquid form 10b, or an amalgam 10 of the substantially cream form 10a and the substantially liquid form 10b in an unequal ratio.

When the amalgam 10 of the antifungal medication is applied to the cold sore 13 during the initial stages of inflammation, the individual may expect the development of the cold sore 13 to cease and subside within approximately three hours, although the amount of time may vary. If the cold sore 13 has already fully developed, then the individual may expect the cold sore 13 to begin healing within approximately twelve hours of initial application of the amalgam 10 of antifungal medication, although the amount of time may vary. Preferably, this regiment should be repeated approximately four times on the first day of treatment and then approximately twice a day until the cold sore 13 has subsided.

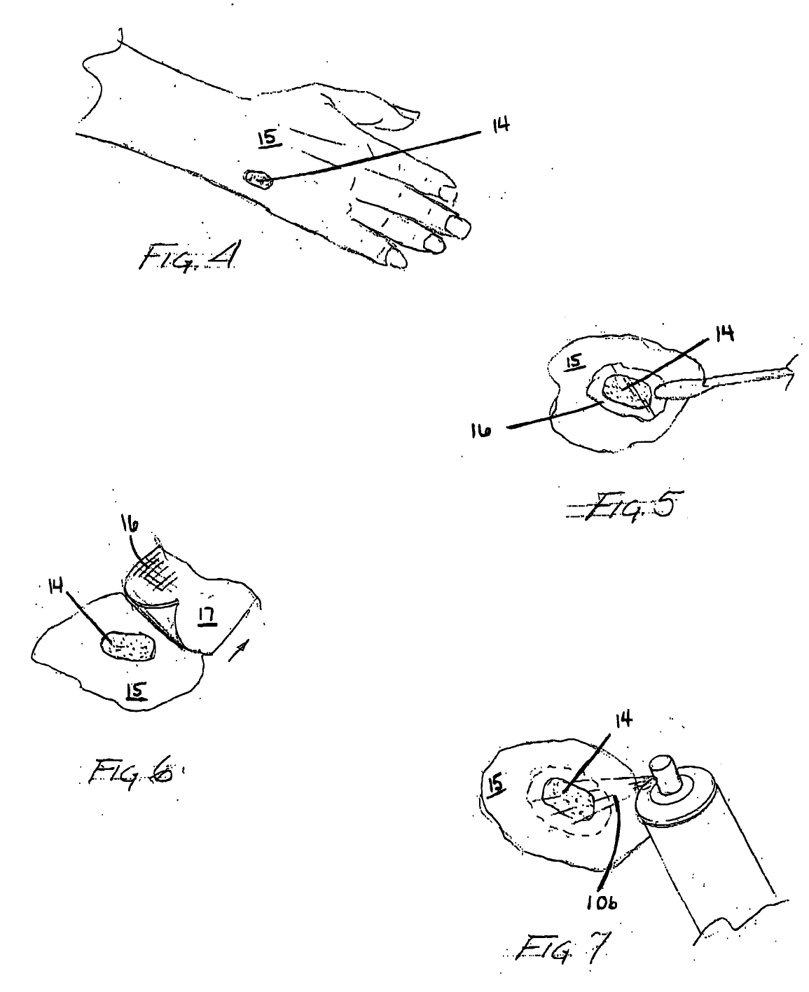

Some pimples may be hard and filled with a clear, yellow liquid. The inventor believes that these particular types of pimples contain a fungal granuloma that cannot be easily removed by squeezing. Therefore, the inventor has described a method of treating fungal pimples 14 with antifungal medication (see FIGS. 4-7). These fungal pimples 14 may appear anywhere on an individual's body, such as on a hand 15 (see FIG. 4). As shown in FIG. 5, the fungal pimple 14 may first be softened with a softening agent 16. This softening agent 16 may be petroleum jelly, such as Vaseline, water, or some other compound capable of softening the fungal pimple 14.

Preferably, after waiting approximately fifteen minutes for the softening agent 16 to soften the fungal pimple 14, the softening agent 16 should then be wiped off (preferably with a clean cloth or towel 17 (see FIG. 6)). Finally, the antifungal medication, in either the substantially cream form 10a (not shown), the substantially liquid form 10b, or an amalgam thereof should be applied to the fungal pimple 14.

Preferably, this regiment should be repeated approximately twice a day and the individual may expect the fungal pimple 14 to heal within approximately three days, although the amount of time may vary.

While the invention has been particularly shown and described with reference to preferred embodiments thereof, it will be understood by those skilled in the art that the foregoing and other changes in form and details may be made therein without departing from the spirit and scope of the invention. It should be understood by those skilled in the art that substantial benefit may also be obtained by using a predetermined amount of the amalgam of antifungal medication that is available in pre-packaged form and is applied with a pre-packaged applicator. It should also be understood by those skilled in the art that further substantial benefit may be received by ingesting a pill form of antifungal medication or by injecting a liquid form of antifungal medication into the infected skin. And it should be understood by those skilled in the art that substantial benefit may be obtained by first cleaning and drying the cold sore or fungal pimple prior to application of the antifungal medication.

1. A method for treating herpes simplex virus comprising the steps of:
providing an antifungal medication; and
applying the antifungal medication onto the infected area, thereby preventing further inflammation and spreading of the virus.

2. The method of claim 1 wherein the herpes simplex virus is herpes simplex virus type one.

3. The method of claim 1 wherein the herpes simplex virus is herpes simplex virus type two.

4. The method of claim 1 wherein the antifungal medication is in a substantially cream form.

5. The method of claim 1 wherein the antifungal medication is in a substantially liquid form.

6. The method of claim 1 wherein the antifungal medication is an amalgam of a substantially cream form and a substantially liquid form.

7. The method of claim 1 wherein the antifungal medication is terbinafine hydrochloride.

8. The method of claim 1 wherein the antifungal medication is LAMISIL^{AT}.

9. The method of claim 1 wherein the infected area is a cold sore.

10. The method of claim 1 wherein the infected area is an individual's genital region.

11. The method of claim 1 wherein the step of applying the antifungal medication further comprises the step of spreading the antifungal medication onto the infected area with one of an applicator and a cotton swab.

12. The method of claim 6 wherein the amalgam is available in a predetermined quantity.

13. A method for treating a fungal pimple comprising the steps of:
softening the fungal pimple; and
applying antifungal medication to the fungal pimple.

14. The method of claim 13 wherein the step of softening the fungal pimple further comprises the steps of:
applying a softening agent to the fungal pimple;
waiting a predetermined amount of time; and
removing the softening agent.

15. The method of claim 13 wherein the antifungal medication is in a substantially cream form.

16. The method of claim 13 wherein the antifungal medication is in a substantially liquid form.

17. The method of claim 13 wherein the antifungal medication is terbinafine hydrochloride.

18. The method of claim 13 wherein the antifungal medication is LAMISIL^{AT}.

19. The method of claim 14 wherein the softening agent is one of water and petroleum jelly.

20. The method of claim 14 wherein the predetermined amount of time is approximately fifteen minutes.
